(19) United States
(12) Plant Patent Application Publication
(10) Pub. No.: US 2014/0245509 P1
(43) Pub. Date: Aug. 28, 2014

(54) Mint plant "Chelan Mint"

(50) ;

(71) Applicant: ESSEX LABORATORIES, INC., Napavine, WA (US)

(72) Inventor: Donald D. Roberts, Independence, OR (US)

(73) Assignee: ESSEX LABORATORIES, INC., Napavine, WA (US)

(21) Appl. No.: 13/815,404

(22) Filed: Feb. 27, 2013

(51) Int. Cl. A01H 5/00

(52) U.S. Cl. PLT/259

(57) ABSTRACT

Mint selection 08-6-10, denominated “Chelan Mint,” is a new Mentha piperita cultivar that produces an essential oil different in composition than commercially grown mint varieties. The essential oil is similar to standard peppermint mint oil in component composition but differs in the typical ratio of components. Organoleptically it differs from typical peppermint oil. It is resistant to mint rust and more resistant to Verticillium mint wilt than current commercially grown varieties.

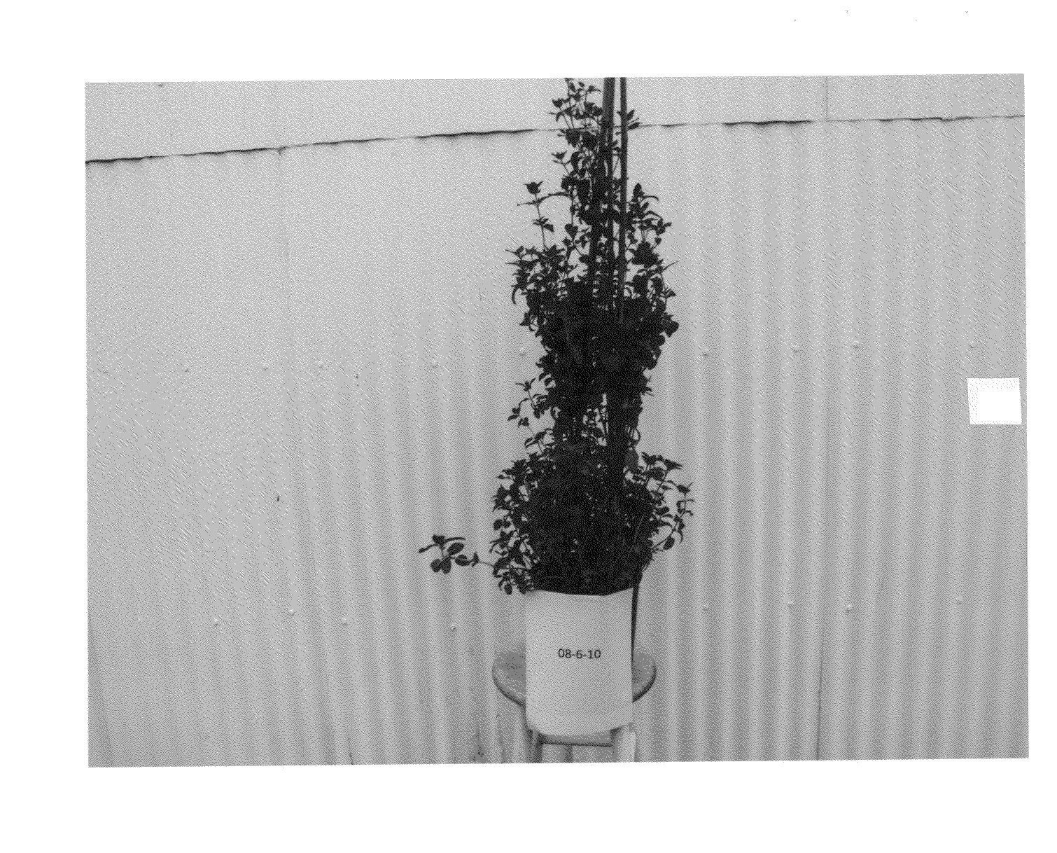

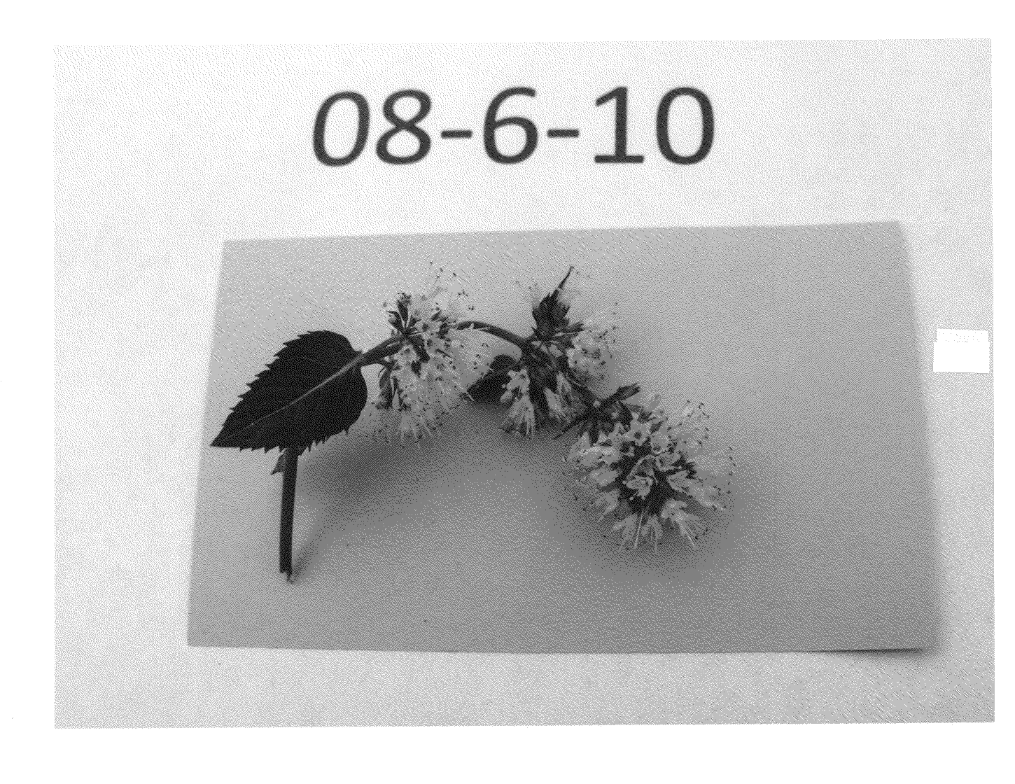

### FEDERAL SPONSORSHIP

None

### TYPE OF PLANT AND NAME OF VARIETY

The present invention relates to a new and distinct variety of peppermint plant developed from a parent of the species Mentha piperita L. The new variety will be identified as “Chelan Mint.”

### SUMMARY OF THE INVENTION

Mint selection 08-6-10, denominated “Chelan Mint,” is a new Mentha piperita cultivar that produces an essential oil different in composition than commercially grown mint varieties. The essential oil is similar to standard peppermint mint oil in component composition but differs in the typical ratio of components. Organoleptically it differs from typical peppermint oil. It is resistant to mint rust and more resistant to Verticillium mint wilt than current commercially grown varieties.

### DISCOVERY AND ASEXUAL REPRODUCTION

This plant was selected from a population of mint seedlings in research plots on land near Monmouth, Oreg. and initially identified as 08-6-10. Selection 08-6-10 originated as a seedling from an open pollinated polyploid ‘Black Mitcham’ parent. Diploid Black Mitcham peppermint is sterile and only becomes fertile in the polyploidy state. The Black Mitcham parent to 08-6-10 was one of several mint lines in a polycross breeding system composed of selected male and female fertile genotypes.

The primary objective in this breeding program was to develop a Mitcham type peppermint variety having a specific oil composition, acceptable yield and resistant to mint wilt and mint rust. Mint wilt is caused by a soil-borne fungus, Verticilium dahlia and mint rust is a leaf infection caused by the air-borne fungus Puccinia menthae. Only seedlings that showed no symptoms of mint wilt or rust were selected for further evaluation. Selection 08-6-10 was vegetative propagated to increase the selection to 20 plants for continued evaluation in 2009. The selection was again vegetative propagated to over 300 plants for planting and evaluation in 2010. When compared to Black Mitcham as a control, 08-6-10 had less (or no) symptoms of mint wilt than the control and had no mint rust infection. Yield of 08-6-10 is equivalent to Black Mitcham in test plots as a single plant and in larger plot evaluations from 2009 to 2010.

Selection 08-6-10 is asexually propagated to maintain the cultivar's genetic integrity and as a means of increasing the selection for commercial planting. Asexual propagation, by tip cuttings or stolon sections, is a common practice in commercial mint cultivation and serves as a means of propagating the normally sterile mint plant. Premier Botanicals has conducted asexual propagation of 08-6-10 for greenhouse and field planting in Monmouth, Oreg., each year since 2008 and the genotype comes true to form with each generation.

### BRIEF DESCRIPTION OF THE PHOTOGRAPHS

The accompanying color photographs show typical greenhouse vegetative growth of 08-6-10 and depicts the color as nearly as reasonably possible.

Photographs 1 and 2 illustrate the growth and flowering pattern of my new mint plant in accordance with the present invention.

Photograph 1 illustrates the growth and branching pattern of the new mint plant.

Photograph 2 illustrates the shape of the leaf and flower on a terminal flowering shoot.

### DESCRIPTION OF PLANT

My new mint plant improves upon and is distinct from other mint plants in several characteristics, including but not limited to, the following:

1. The ability to produce an essential oil different in composition but with similar components as ‘Black Mitcham’ peppermint;

2. A more compact plant but with a branching pattern similar to its parent;

3. An early spring growth for a potential double harvest in one growing season;

4. A level of resistance to mint wilt (Verticillium dahliae) equal to or greater than that of its parent and the commercially grown Black Mitcham peppermint.

5. Resistance to mint rust caused by Puccinia menthae.

The essential oil extracted from 08-6-10 has the same components as that of commercial oil produced by Black Mitcham peppermint as illustrated in Table 1. However, the ratio of oil components differs in the commercial peppermint oil and that of 08-6-10. The concentration of menthone in the oil of 08-6-10 is lower than that of Black Mitcham. The amount of Menthofuran and Menthyl Acetate in the oil of 08-6-10 is higher than that of Black Mitcham. The menthol has a similar concentration as that of Black Mitcham. Organoleptically the oil of 08-6-10 is different than that of Black Mitcham, reflecting the difference in oil component ratios.

TABLE 1
Composition of 08-6-10 Peppermint Oil from Test Plots Compared to
commercial Black Mitcham Peppermint Oil and Oil from Black Mitcham
Peppermint Grown in Test Plots located near Monmouth, Oregon.

| | Commercial | 2008 Test Plot | 2008 Test Plot |
|---|---|---|---|
| | Black Mitcham 1/ | Black Mitcham 2/ | 08-6-10 3/ |
| α-pinene | 0.82 | 0.80 | <1.0 |
| β-pinene | 1.06 | 1.03 | <1.0 |
| 1-limonene | 1.78 | 1.73 | 1.4 |
| 1,8-cineole | 5.33 | 4.90 | 4.2 |
| 1-menthone | 20.00 | 19.43 | 10.0 |
| Menthofuran | 4.35 | 4.23 | 20.7 |
| Iso-menthone | 3.19 | 3.14 | 1.3 |
| 1-menthyl acetate | 5.09 | 5.36 | 7.8 |
| Neo-menthol | 5.40 | 5.52 | 4.0 |
| B-caryophyllene | 0.83 | 0.85 | 1.6 |
| 1-menthol | 45.20 | 44.60 | 44.2 |
| Pulegone | 2.17 | 2.14 | <1.0 |
| Germacrene-D | 1.22 | 1.37 | <1.0 |

The numbers listed in the above table are percentages based upon the analysis of the respective mint oils by gas chromatography. The percentages are determined by calculation of the relative peak areas.
1/ Commercial oil sample typical of what is produced by peppermint producers.
2/ Black Mitcham oil from a control plant in the 2008 seedling test plot.
3/ 08-6-10 oil from a single plant harvest from the 2008 test plots.

### TAXONOMIC DESCRIPTION OF 08-6-10

This new plant, under greenhouse and field growing conditions, is a bush type plant with lateral branches at each node of the main stems. The growth of 08-6-10 in height is 10 to 20% more than Black Mitcham growing under similar conditions but will vary based on fertilizer, soil quality, and water application, amongst other known factors that affect growth patterns. Secondary and tertiary branching occurs to form a compact growth habit. When 08-6-10 is mature and ready for harvest, the main stem at mid-plant (approximately between the eleventh and twelfth node) is 3.3-3.9 mm in width. The secondary and tertiary branch stems are 2.2-2.7 mm and 1.5-2.0 mm in width, respectively.

Mature leaves at the bottom of the plant are ovate to lanceolate as are leaves on secondary branch stems. Leaves on upper mature plants, both main and secondary stems are more lanceolate (Photograph 2). Mid-main stem leaf size at flowering is 24-30 mm in width and 55-65 mm in length. Leaf size on secondary branches at flowering is 15-18 mm in width and 27-32 mm in length. Leaf petioles on the main stem leaves are 11-15 mm in length while petioles on secondary branch stem leaves are 4-6 mm in length. Leaves on the mid-main stem and lower tend to be slightly lobed and denticulate while the leaves on the upper plant tend to be more dentate. The main stem leaves have from 9 to 12 teeth on each side. The leaf is dark green in color, ranging from Royal Horticulture Society Color Code N137B to N137C in the green group classification. The leaf has 6-9 lateral veins, more or less in parallel off the main vain that runs from the petiole to the tip of the leaf. The veins are prominent in all leaves of 08-6-10.

The inflorescence is an inconspicuous spike with capitate flowers developing at the last few nodes of the stem. The capitate flowers are 15-20 mm in width and 10-15 mm in length. The flowers consist of five petals fused into a two lipped corolla. The corolla is light in color ranging from Royal Horticulture Society Color Code 84D to white in the Violet Group. The Calyx is generally green and is 143B to 143C RHS Color Code, Green Group. The gynoecium consists of a single pistil with two lobed stigma that is exserted. The androecium consists of four stamens, each with a distinct filament and anther.

While the plant that comprises the present invention has been described in connection with a specific embodiment thereof, it will be understood that this application is intended to cover any variation, uses, or adaptation of the invention (particular those induced by cultivation under different environmental conditions) following, in general, the principles of the invention and including such departures from the present disclosure as come within known or customary practice in the art to which the invention pertains and as may be applied to the essential features hereinbefore set forth, and as fall within the scope of the invention and the limits of the appended claim.

I claim:

1. A new and distinct variety of peppermint plant, substantially as shown and described, characterized particularly by improving resistance to mint rust, and producing a unique essential oil.
